(19) United States
(12) Patent Application Publication
(10) Pub. No.: US 2008/0233579 A1
(43) Pub. Date: Sep. 25, 2008

(54) PRIMER SET FOR DETECTING OVEREXPRESSION OF KATP CHANNEL AND KIT COMPRISING SAID PRIMER SET

(76) Inventors: Won Sun Park, Busan (KR); Na Ri Kim, Busan (KR); Jae-Hong Ko, Cheju (KR); Jin Han, Busan (KR)

Correspondence Address: CHRISTIE, PARKER & HALE, LLP, PO BOX 7068, PASADENA, CA, 91109-7068, US

(21) Appl. No.: 11/965,687

(22) Filed: Dec. 27, 2007

(30) Foreign Application Priority Data
Dec. 29, 2006 (KR) 10-2006-0137490

(51) Int. Cl. C12Q 1/68; C07H 21/00

(52) U.S. Cl. 435/6; 536/24.33

(57) ABSTRACT

The present invention relates to a primer set for confirming an increase of mRNA in an ATP-sensitive potassium channel (K_{ATP} channel)(Kir6.1) having an effect of protecting heart from hypoxia or an ischemic disease; a kit including the primer set; and a method of identifying an agent for treating an ischemic heart disease.

### CROSS-REFERENCE TO RELATED APPLICATION

This application claims priority to and the benefit of Korean Patent Application No. 10-2006-0137490 filed in the Korean Intellectual Property Office on Dec. 29, 2007, the entire content of which is incorporated herein by reference.

### FIELD OF THE INVENTION

The present invention relates to a method of diagnosing an increase in mRNA of an ATP-sensitive potassium channel (K_{ATP} channel)(Kir6.1) having an effect of protecting heart from hypoxia or an ischemic disease, and a kit for use in the method. Such a method and a kit are very useful in developing an agent for clinically diagnosing or treating the ischemic disease.

### BACKGROUND OF THE INVENTION

Heart is seriously damaged when oxygen is not smoothly provided into a heart cell due to a surgical operation or a heart disease inducing hypoxia, etc. A body protecting mechanism occurs internally against any damage of a human body. The representative example of such body protecting mechanism against the heart damage is an activation of a mitochondrial K_{ATP} channel. The opening of the K_{ATP} channel mediates cardioprotective effects induced by ischemic preconditioning, heat shock and pharmaceutical agents such as non-toxic derivatives of adenosine, acetylcholine (Ach), opioid and endotoxin monophosphoryl lipid.

It has been observed that inducing lethal myocardial ischemia for a long period after inducing myocardial ischemia and reperfusion repeatedly for a short period causes less damage to myocardia compared to inducing lethal myocardial ischemia immediately without such preconditioning. Such a phenomenon is called as cardioprotective effect by ischemic preconditioning (Murray and Jenning, 1986).

The mechanism underlying the cardioprotective effect has been initially postulated as that opening of sarcK_{ATP} channel enhances the shortening of action potential duration, which causes reduction in Ca²⁺ entry into cells and prevention of Ca²⁺ overload, thereby inhibiting the necrosis of myocardial cells. However, later studies have proved that shortening of action potential duration is not prerequisite for cardiac protection by ischemic preconditioning, and the effect of ischemic preconditioning is maintained even when action potential duration is prolonged by other potassium channel blockers than that for the K_{ATP} channel. It was recently suggested that the mitoK_{ATP} channel is involved in the ischemic preconditioning as an important effector, based on some evidences showing that the opening of mitoK_{ATP} channel causes the effect of the ischemic preconditioning and, however, the cardioprotective effect of the ischemic preconditioning is abolished by blocking mitoK_{ATP} channel with 5-hydroxydecanoate (5-HD). Further, the effect of the ischemic preconditioning was not abolished when sarcK_{ATP} channel was blocked by adding HMR1098 as an optional blocker for the sarcK_{ATP} channel. Accordingly, it is recognized that the action of the mitoK_{ATP} channel is the most important part in the ischemic preconditioning. It is known that the opening of the mitoK_{ATP} channel leads to the cardioprotective effect through the inhibition of Ca²⁺ influx. The administration of mitoK_{ATP} channel blocker such as 5-hydroxydecanoate (5-HD) fails to inhibit the Ca²⁺ overload within the mitochondria so that the protecting effect by the ischemic preconditioning is not exerted. The protection of mitochondrial function is engaged with the generation of intracellular ATP, and ATP-dependent Na⁺-K⁺ ATPase plays a very crucial role in releasing Na⁺ accumulated within a cell. Otherwise, Ca²⁺ overload occurs in a cell by exchanging Na⁺ with Ca²⁺, leading to death of the cell. In such sense, the opening of the mitoK_{ATP} channel can be very crucial in preventing the Ca²⁺ overload in mitochondria, thereby keeping the function of mitochondria better.

Further, the mitochondrial K_{ATP} channel in a myocardial cell participates in anti-arrhythmia and anti-cerebral infarction effect of a K_{ATP} activating agent during ischemia and reperfusion.

Such a mitochondrial K_{ATP} channel is not elucidated yet at its genetic level (gene cloning), however, is assumed to be expressed in mitochondrial membrane in the form of subtypes of Kir6.1 or Kir6.2.

As described previously, it is reported that the mitochondrial K_{ATP} channel is activated as a form of cell defense mechanism, thereby providing heart-protecting effect. However, the presence of the mitochondrial K_{ATP} channel can be just indirectly estimated since the genetic essence of the mitochondrial K_{ATP} channel is not elucidated. Further, most experiments on the mitochondrial K_{ATP} channel have fatal weak point that a complex technology and an expensive equipment such as a patch clamp or a confocal image are required.

### SUMMARY OF THE INVENTION

It is one object of the present invention to provide an experimental basis, e.g., for diagnosing a heart disease or developing a therapeutic agent for an ischemic disease by providing a method capable of confirming the expression of such a mitochondrial K_{ATP} channel at its mRNA level with RT-PCR, and a kit for use in the method.

It is another object of the present invention to provide a method and a kit that can be utilized easily and inexpensively in the basic/clinical medical field dealing with an ischemic disease, as well as the screening of a therapeutic agent for such disease, and biotechnological researches.

The constitutions of the present invention for accomplishing the above objects of the present invention are as follows.

The present invention provides a primer set for confirming an increase of mRNA in an ATP-sensitive potassium channel (K_{ATP} channel) (Kir6.1) having an effect of protecting heart from hypoxia or an ischemic disease; a kit including the primer set; and a method of identifying an agent for treating an ischemic heart disease by using the primer set.

In one aspect of the present invention, there is provided a primer set for detecting the overexpression of a mitochondrial K_{ATP} channel having the following sequences:

| | |
|---|---|
| A forward primer: | |
| 5′-ATCCCGGAGGAGTATGTGCT-3′ | (SEQ ID NO: 1) |
| and | |
| A reverse primer: | |
| 5′-CGTGAATGACCTGACATTGG-3′. | (SEQ ID NO: 2) |

In another aspect of the present invention, there is provided a kit for confirming the overexpression of the mitochondrial K_{ATP} channel comprising a primer set for reverse transcriptase-polymerase chain reaction (RT-PCR) consisting of a forward primer having the sequence of SEQ ID NO: 1 and a reverse primer having the sequence of SEQ ID NO: 2. The kit according to the present invention can include a conventional polymerase, a DNTP mixture, etc. required in the polymerase chain reaction.

In yet another aspect of the present invention, there is provided a kit for diagnosing ischemic preconditioning comprising a primer set for reverse transcriptase-polymerase chain reaction consisting of the forward primer having a sequence of SEQ ID NO: 1 and the reverse primer having a sequence of SEQ ID NO: 2.

In still yet another aspect of the present invention, there is provided a method of identifying an agent for treating a disease associated with the expression of the mitochondrial K_{ATP} channel by utilizing the forward primer having the sequence of SEQ ID NO: 1 and the reverse primer having the sequence of SEQ ID NO: 2.

In still yet another aspect of the present invention, there is provided a method for identifying a therapeutic agent for a mitochondrial K_{ATP} channel-related ischemic heart disease comprising: subjecting a mitochondrial cell in the presence of said agent or in the absence of said agent; amplifying the mRNA of mitochondrial K_{ATP} channel with the forward primer having the sequence of SEQ ID NO: 1 and the reverse primer having the sequence of SEQ ID NO: 2; and comparing the expressed mRNA level of mitochondrial K_{ATP} channel in the presence of said agent with the expressed mRNA level in the absence of said agent.

In still yet another aspect of the present invention, there is provided a method for identifying a therapeutic agent for arrhythmia or cerebral infarction comprising: subjecting a mitochondrial cell in the presence of said agent or in the absence of said agent; amplifying the mRNA of mitochondrial K_{ATP} channel with the forward primer having the sequence of SEQ ID NO: 1 and the reverse primer having the sequence of SEQ ID NO: 2; and comparing the expressed mRNA level of mitochondrial K_{ATP} channel in the presence of said agent with the expressed mRNA level in the absence of said agent. A therapeutic agent useful for treating an ischemic heart disease, arrhythmia or cerebral infarction, etc. can be effectively identified or screened by subjecting an ischemic heart model commonly used in the art in the presence or in the absence of a candidate therapeutic agent for an ischemic heart disease, arrhythmia or cerebral infarction, etc.; performing RT-PCR by utilizing the primer set according to the present invention, thereby determining the amount of the expressed mitochondrial K_{ATP} channel; and comparing the amount of the expressed mitochondrial K_{ATP} channel in the presence of the candidate therapeutic agent with the amount of the expressed mitochondrial K_{ATP} channel in the absence of the candidate therapeutic agent.

### BRIEF DESCRIPTION OF THE DRAWINGS

The above objects and features of the present invention will become more apparent from the following description of the preferred embodiments given in conjunction with the accompanying drawings, in which:

FIG. 1 is a result of reverse transcriptase-polymerase chain reaction (RT-PCR) that compares the amount of the expressed K_{ATP} channel by utilizing a total of RNA isolated from the heart cell of a normal mouse (non-treated group, Cont) and a mouse (treated group, IPC) whose ischemic preconditioning is induced through perfusion following removing the heart.

FIG. 2 illustrates a Table that calculates a relative expression ratio of mRNA obtained by dividing the amount of mRNA of K_{ATP} channel of a non-treated group and a treated group amplified through polymerase chain reaction (PCR) by the amount of the expressed mRNA of a housekeening gene (β-actin).

### DETAILED DESCRIPTIONS OF THE PREFERRED EMBODIMENTS

In the following detailed description, reference is made to the accompanying drawings that show, by way of illustration, specific embodiments in which the present invention may be practiced. These embodiments are described in sufficient detail to enable those skilled in the art to practice the present invention. It is to be understood that the various embodiments of the present invention, although different from one another, are not necessarily mutually exclusive. For example, a particular feature, structure, or characteristic described herein in connection with one embodiment may be implemented within other embodiments without departing from the spirit and scope of the present invention. In addition, it is to be understood that the location or arrangement of individual elements within each disclosed embodiment may be modified without departing from the spirit and scope of the present invention. The following detailed description is, therefore, not to be taken in a limiting sense, and the scope of the present invention is defined only by the appended claims, appropriately interpreted, along with the full range of equivalents to which the claims are entitled. In the drawings, like numerals refer to the same or similar functionality throughout the several views.

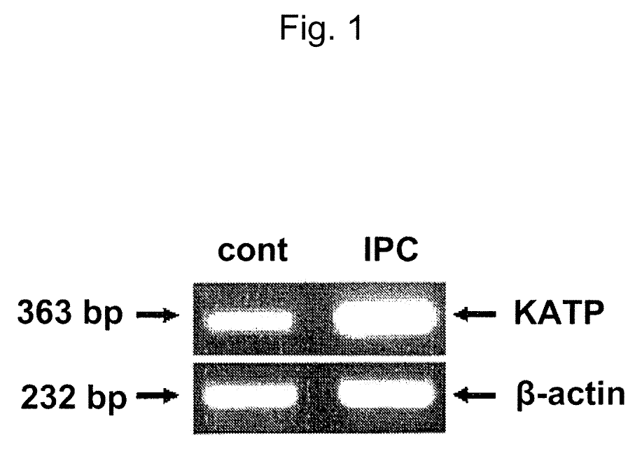

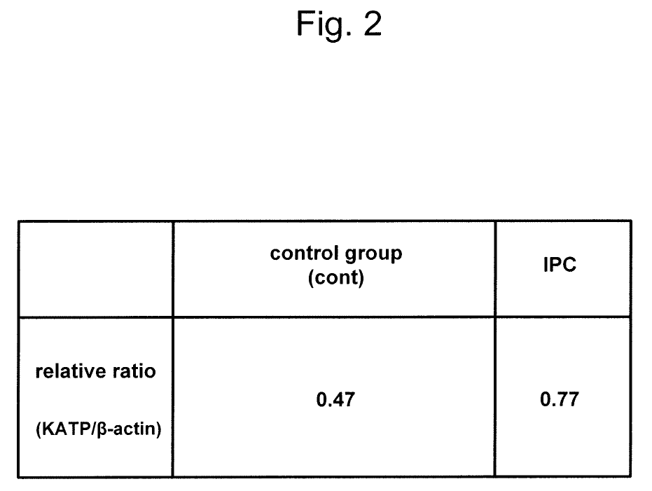

The overexpression of a K_{ATP} channel (Kir6.1) mRNA induced by ischemic preconditioning in heart was determined through PCR. For this experiment, a heart is firstly removed from a mouse, and a perfusion solution is flowed into the coronary artery of the heart by utilizing Langendorff system, which is generally used in flowing a perfusion solution into a heart. The heart was two times repeatedly perfusioned with an ischemic solution (a normal Tyrode solution containing less than 5% pO2) (5 minutes) and then with a normal Tyrode solution (containing more than 20% pO2) (5 minutes), thereby inducing ischemic preconditioning status. The heart subjected to perfusion with the normal Tyrode solution only was set as a control group (indicated as “Cont” in FIGS. 1 and 2), and the heart of a mouse, on which ischemic preconditioning was induced, was set as a treated group (indicated as “IPC” in FIGS. 1 and 2). Total RNA was isolated from the respective heart tissues in a manner typically performed in a laboratory, cDNA was synthesized by utilizing a reverse transcriptase and oligo (dT), PCR was performed for 30 cycles, and then the expressed amount of the amplified K_{ATP} channel mRNA was divided by the expressed mRNA amount of the housekeeping gene (actin in FIGS. 1 and 2) to calculate the relative difference in the expressed amount in the respective groups (FIG. 2).

The composition of the PCR solution was as follows:

| | | |
|---|---|---|
| 10 x BD advantage 2 PCR buffer | 2 | μl |
| 2.5 mM dNTP mixture | 1 | μl |
| Forward primer of SEQ ID NO: 1 | 0.5 | μl |
| (10 pmol/μl) | | |
| Reverse primer of SEQ ID NO: 2 | 0.5 | μl |
| (10 pmol/μl) | | |
| cDNA template | 1 | μl |
| DNA polymerase | q.s. | (μl) |
| Distilled water | (13 - DNA polymerase) | μl |

The band obtained by utilizing the primer set constructed for amplifying the mRNA of the K_{ATP} channel was collected to analyze its sequence. It was confirmed that the analyzed sequence completely conforms with the sequence of Kir6.1.

In the experiment, the RT-PCR was conducted by employing a PCR apparatus or a reagent (e.g., reverse transcriptase, polymerase, agarose, etc.) typically used in laboratories. The primers constructed for amplifying the mRNA of the K_{ATP} channel were designed to target any parts showing 100% consensus by comparing and examining the homology of Kir6.1 between several mammals. Optimum primer pairs were selected to perform the experiment. Among these, most superior and stable primer pair below was utilized in the present invention as follows:

| | |
|---|---|
| a forward primer: | |
| 5′-ATCCCGGAGGAGTATGTGCT-3′; | (SEQ ID NO: 1) |
| a reverse primer: | |
| 5′-CGTGAATGACCTGACATTGG-3′; | (SEQ ID NO: 2) |
| and | |
| annealing temperature: 60° C. | |

More accurate comparison was performed by evaluating relative expression level of β-actin, which is a housekeeping gene. The primer pair of the β-actin used for the experiment was as follows:

| | |
|---|---|
| a forward primer: | |
| 5′-CATTGTGATGGACTCCGGAGACGG-3′; | (SEQ ID NO: 3) |
| a reverse primer: | |
| 5′-CATCTCCTGCTCGAAGTCTAGAGC-3′; | (SEQ ID NO: 4) |
| and | |
| annealing temperature: 56° C. | |

### EFFECTS FROM PRACTICING THE PRESENT INVENTION

The present invention relates to a diagnosing method capable of quickly examining at gene level the process that a heart protects and recovers itself from an ischemic damage, and is useful for development of a therapeutic agent for an ischemic heart disease, or clinical diagnosis and prevention for the ischemic heart disease.

While the present invention has been shown and described with respect to the preferred embodiments, it will be understood by those skilled in the art that various changes and modifications may be made without departing from the spirit and the scope of the present invention as defined in the following claims.

What is claimed is:

1. A primer set for detecting the overexpression of a mitochondrial K_{ATP} channel consisting of a forward primer having the sequence of SEQ ID NO: 1 and a reverse primer having the sequence of SEQ ID NO: 2.

2. A kit for detecting the overexpression of a mitochondrial K_{ATP} channel comprising a primer set for reverse transcriptase-polymerase chain reaction (RT-PCR) consisting of a forward primer having the sequence of SEQ ID NO: 1 and a reverse primer having the sequence of SEQ ID NO: 2.

3. A kit for diagnosing ischemic preconditioning comprising a primer set for reverse transcriptase-polymerase chain reaction (RT-PCR) consisting of a forward primer having the sequence of SEQ ID NO: 1 and a reverse primer having the sequence of SEQ ID NO: 2.

4. A method for identifying a therapeutic agent for a mitochondrial K_{ATP} channel-related ischemic heart disease comprising: subjecting a mitochondrial cell in the presence of said agent or in the absence of said agent; amplifying the mRNA of mitochondrial K_{ATP} channel using a forward primer of having the sequence of SEQ ID NO: 1 and a reverse primer having the sequence of SEQ ID NO: 2; and comparing the expressed mRNA level of mitochondrial K_{ATP} channel in the presence of said agent with the expressed mRNA level in the absence of said agent.

5. A method for identifying a therapeutic agent for anti-arrhythmia or anti-cerebral infarction comprising: subjecting a mitochondrial cell in the presence of said agent or in the absence of said agent; amplifying the mRNA of mitochondrial K_{ATP} channel using a forward primer of having the sequence of SEQ ID NO: 1 and a reverse primer having the sequence of SEQ ID NO: 2; and comparing the expressed mRNA level of mitochondrial K_{ATP} channel in the presence of said agent with the expressed mRNA level in the absence of said agent.


SEQUENCE LISTING (4 sequences)

SEQ ID NO: 1 - DNA, 20 bp, Artificial Sequence
  Forward primer
atcccggaggagtatgtgct

SEQ ID NO: 2 - DNA, 20 bp, Artificial Sequence
  Reverse primer
cgtgaatgacctgacattgg

SEQ ID NO: 3 - DNA, 24 bp, Artificial Sequence
  Forward primer for actin
cattgtgatggactccggagacgg

SEQ ID NO: 4 - DNA, 24 bp, Artificial Sequence
  Reverse primer for actin
catctcctgctcgaagtctagagc